(19) United States
(12) Patent Application Publication
(10) Pub. No.: US 2022/0022953 A1
(43) Pub. Date: Jan. 27, 2022

(54) ESOPHAGUS CATHETER FOR IRREVERSIBLE ELECTROPORATION

(71) Applicant: Boston Scientific Scimed, Inc., Maple Grove, MN (US)

(72) Inventors: Allan C. Shuros, St Paul, MN (US); Brendan E. Koop, Ham Lake, MN (US)

(21) Appl. No.: 17/382,967

(22) Filed: Jul. 22, 2021

Related U.S. Application Data
(60) Provisional application No. 63/056,296, filed on Jul. 24, 2020.

(51) Int. Cl. A61B 18/14

(52) U.S. Cl. CPC A61B 18/1492; A61B 2018/00613

(57) ABSTRACT

At least some embodiments of the present disclosure are directed to an electroporation ablation device having a first catheter and a second catheter. The first catheter comprises one or more first electrodes and has a first surface area. The second catheter comprises one or more second electrodes and has a second surface area. When the electroporation ablation device is in operation for ablating a target tissue, the first catheter is configured to be disposed in an extracardiac location and anatomically proximate to the target tissue, the second catheter is configured to be disposed at an intracardiac location proximate to the target tissue, and the electroporation ablation device is configured to generate an electric field between the one or more first electrodes and the one or more second electrodes with electric field strength sufficient to ablate the target tissue via irreversible electroporation.

### CROSS REFERENCE TO RELATED APPLICATION

This application claims priority to Provisional Application No. 63/056,296, filed Jul. 24, 2020, all of which are herein incorporated by reference in their entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells in order to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kW targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells.

### SUMMARY

As recited in examples, Example 1 is an electroporation ablation device. The electroporation ablation device comprises a first catheter and a second catheter. The first catheter comprises one or more first electrodes and has a first surface area. The second catheter comprises one or more second electrodes and has a second surface area. The first surface area is larger than the second surface area. When the electroporation ablation device is in operation for ablating a target tissue, the first catheter is configured to be disposed in an esophagus and anatomically proximate to the target tissue, the second catheter is configured to be disposed at an intracardiac location proximate to the target tissue, and the electroporation ablation device is configured to generate an electric field between the one or more first electrodes and the one or more second electrodes with electric field strength sufficient to ablate the target tissue via irreversible electroporation.

Example 2 is the electroporation ablation device of Example 1, wherein the first catheter comprises a temperature sensor.

Example 3 is the electroporation ablation device of Example 2, wherein the temperature sensor is configured to detect a temperature in the esophagus when the electroporation ablation device is in operation.

Example 4 is the electroporation ablation device of Example 3, wherein the electric field strength is reduced when the detected temperature is greater than a predetermined threshold.

Example 5 is the electroporation ablation device of any one of Examples 1-4, wherein the first catheter is deflectable.

Example 6 is the electroporation ablation device of Example 1, wherein the one or more first electrodes are configured to provide a return path for an ablative energy delivered to the electroporation ablation device.

Example 7 is the electroporation ablation device of any one of Examples 1-6, wherein the one or more second electrodes comprise a plurality of distal electrodes and a plurality of proximal electrodes, and wherein the plurality of distal electrodes are disposed closer to a distal end of the second catheter than the plurality of proximal electrodes.

Example 8 is the electroporation ablation device of any one of Examples 1-7, wherein the first surface area is larger than the second surface area by 10% of the second surface area.

Example 9 is the electroporation ablation device of any one of Examples 1-8, wherein the first catheter comprises an inflatable balloon.

Example 10 is the electroporation ablation device of any one of Examples 1-9, wherein the electric field strength is less than 1500 volts per centimeter.

Example 11 is the electroporation ablation device of any one of Examples 1-10, wherein the first catheter comprises a plurality of splines, and wherein the one or more first electrodes are disposed on the plurality of splines.

Example 12 is the electroporation ablation device of any one of Examples 1-11, wherein the one or more second electrodes are individually addressable.

Example 13 is a system comprising the electroporation ablation device of any one of Examples 1-12.

Example 14 is the system of Example 13, further comprising: a pulse generator configured to generate and deliver ablative energy to the electroporation ablation device.

Example 15 is the system of Example 14, further comprising: a controller coupled to the pulse generator and the electroporation ablation device and configured to control the ablative energy delivered by the pulse generator.

Example 16 is an electroporation ablation device. The electroporation ablation device includes a first catheter comprising one or more first electrodes and having a first surface area, and a second catheter comprising one or more second electrodes and having a second surface area, where the first surface area is larger than the second surface area. When the electroporation ablation device is in operation for ablating a target tissue, the first catheter is configured to be disposed in an esophagus and anatomically proximate to the target tissue, the second catheter is configured to be disposed at an intracardiac location proximate to the target tissue, and the electroporation ablation device is configured to generate an electric field between the one or more first electrodes and the one or more second electrodes with electric field strength sufficient to ablate the target tissue via irreversible electroporation.

Example 17 is the electroporation ablation device of Example 16, wherein the first catheter comprises a temperature sensor.

Example 18 is the electroporation ablation device of Example 17, wherein the temperature sensor is configured to detect a temperature in the esophagus when the electroporation ablation device is in operation.

Example 19 is the electroporation ablation device of Example 18, wherein the ablative energy is reduced when the detected temperature is greater than a predetermined threshold.

Example 20 is the electroporation ablation device of Example 16, wherein the first catheter is deflectable.

Example 21 is the electroporation ablation device of Example 16, wherein the one or more first electrodes are configured to provide a return path for the ablative energy.

Example 22 is the electroporation ablation device of Example 16, wherein the one or more second electrodes comprise a plurality of distal electrodes and a plurality of proximal electrodes, and wherein the plurality of distal electrodes are disposed closer to a distal end of the second catheter than the plurality of proximal electrodes.

Example 23 is the electroporation ablation device of Example 16, wherein the first surface area is larger than the second surface area by 10% of the second surface area.

Example 24 is the electroporation ablation device of Example 16, wherein the first catheter comprises an inflatable balloon.

Example 25 is the electroporation ablation device of Example 16, wherein the ablative energy is less than 1500 volts per centimeter.

Example 26 is a method using an electroporation ablation device. The method includes the steps of: disposing a first catheter of the electroporation ablation device anatomically approximate to a target ablation location in an extracardiac chamber, the first catheter comprising one or more first electrodes; disposing a second catheter of the electroporation ablation device approximate to the target ablation location in an intracardiac chamber, the second catheter comprising one or more second electrodes; and generating an electric field between the one or more first electrodes and the one or more second electrodes with electric field strength sufficient to ablate the target tissue via irreversible electroporation.

Example 27 is the method of Example 26, wherein the first catheter comprises a temperature sensor.

Example 28 is the method of Example 26, wherein the first catheter is disposed in the esophagus.

Example 29 is the method of Example 28, wherein the electric field strength is reduced when the detected temperature is greater than a predetermined threshold.

Example 30 is the method of Example 26, wherein the first catheter is deflectable.

Example 31 is an electroporation ablation system. The electroporation ablation system includes an electroporation ablation device, a pulse generator configured to generate and deliver ablative energy to the electroporation ablation device, and a controller coupled to the pulse generator and the electroporation ablation device. The electroporation ablation device includes a first catheter comprising one or more first electrodes and having a first surface area, and a second catheter comprising one or more second electrodes and having a second surface area, where the first surface area is larger than the second surface area. When the electroporation ablation device is in operation for ablating a target tissue, the first catheter is configured to be disposed in an esophagus and anatomically proximate to the target tissue, and the second catheter is configured to be disposed at an intracardiac location proximate to the target tissue.

Example 32 is the electroporation ablation system of Example 31, wherein the electroporation ablation device is configured to generate an electric field between the one or more first electrodes and the one or more second electrodes with electric field strength sufficient to ablate the target tissue via irreversible electroporation.

Example 33 is the electroporation ablation system of Example 31, wherein the first catheter is deflectable.

Example 34 is the electroporation ablation system of Example 31, wherein the first surface area is larger than the second surface area by 10% of the second surface area.

Example 35 is the electroporation ablation system of Example 31, wherein the one or more first electrodes are configured to provide a return path for the ablative energy.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 depicts an illustrative system diagram for an electroporation ablation system or device, in accordance with embodiments of the subject matter of the disclosure.

FIG. 2A depicts an illustrative view of an example of an electroporation ablation device in operation, in accordance with embodiments of the subject matter of the disclosure.

FIG. 2B depicts another illustrative view of another example of an electroporation ablation device in operation, in accordance with embodiments of the subject matter of the disclosure.

FIGS. 3A and 3B are diagrams illustrating example embodiments of catheters that can be used for electroporation, including ablation by irreversible electroporation, in accordance with embodiments of the subject matter of the disclosure.

FIG. 4 is an example flow diagram depicting an illustrative method of using an electroporation ablation device, in accordance with some embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

As the terms are used herein with respect to measurements (e.g., dimensions, characteristics, attributes, components, etc.), and ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

Although illustrative methods may be represented by one or more drawings (e.g., flow diagrams, communication flows, etc.), the drawings should not be interpreted as implying any requirement of, or particular order among or between, various steps disclosed herein. However, certain some embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

As used herein, the term “based on” is not meant to be restrictive, but rather indicates that a determination, identification, prediction, calculation, and/or the like, is performed by using, at least, the term following “based on” as an input. For example, predicting an outcome based on a particular piece of information may additionally, or alternatively, base the same determination on another piece of information.

Cryo energy and radio-frequency (RF) energy kill tissues indiscriminately through cell necrosis, which can damage the esophagus, the phrenic nerve, coronary arteries, in addition to other undesired effects. Irreversible electroporation (IRE) uses high voltage, short (ms) pulses to kill cells through apoptosis. IRE can be targeted to kill myocardium, sparing other adjacent tissues including the esophageal vascular smooth muscle and endothelium. The posterior LA wall is embryologically venous tissue and along with the pulmonary veins is high contributor for drivers of atrial tachycardias making it a target for ablation. IRE using unipolar (e.g., catheter tip to cutaneous electrode) configuration generally creates deep lesions but results in extracardiac stimulation of nerves and skeletal muscle. Bipolar configuration reduces this side effect but may have less tissue penetration and be more difficult to achieve transmural lesions. Embodiments of the present disclosure are directed to systems/devices and methods for IRE that are capable of creating transmural lesions while avoiding extracardiac stimulation. In some embodiments, an exploration ablation device having two catheters, one to be disposed in the intracardiac chamber of a patient and one to be disposed in the extracardiac chamber of the patient, is used in such systems and methods.

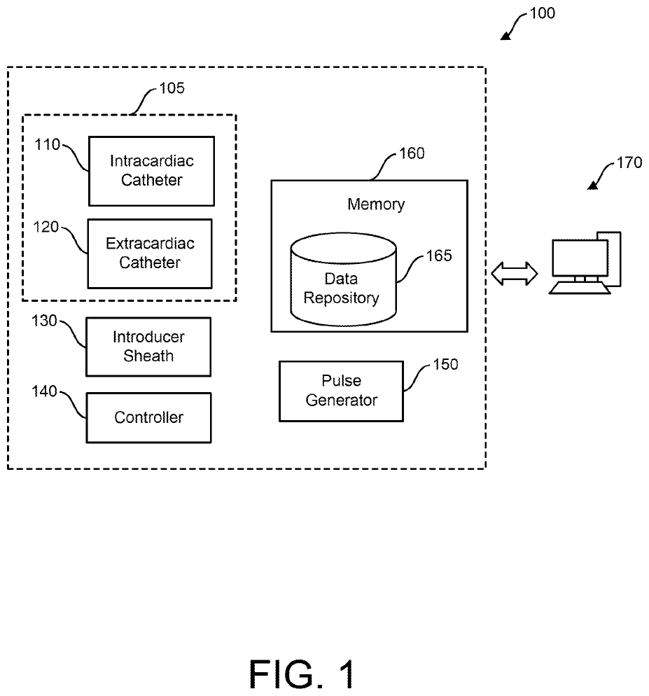

FIG. 1 depicts an illustrative system diagram for an electroporation ablation system or device 100, in accordance with embodiments of the subject matter of the disclosure. The electroporation ablation system/device 100 includes a pair of catheters 105, an introducer sheath 130, a controller 140, a pulse generator 150, and a memory 160. In embodiments, the electroporation ablation system/device 100 is configured to deliver electric field energy to targeted tissue in a patient's heart to create tissue apoptosis, rendering the tissue incapable of conducting electrical signals. In some cases, the electroporation ablation system/device 100 may connect with other system(s) 170, for example, a mapping system, an electrophysiology system, and/or the like.

In embodiments, the pair of catheters 105 includes an intracardiac catheter 110 and an extracardiac catheter 120. The intracardiac catheter 110 is designed to be disposed by a target ablation location in the intracardiac chamber. As used herein, an intracardiac chamber refers to cardiac chamber and its surrounding blood vessels (e.g., pulmonary veins). The extracardiac catheter 120 is designed to be disposed anatomically close to the ablation target in the extracardiac chamber. As used herein, an extracardiac chamber refers to a body internal chamber that is outside of the intracardiac chamber. In some cases, the extracardiac catheter 120 and/or the intracardiac catheter 110 includes one or more sensors (e.g., temperature sensor, location sensor, etc.). In one embodiment, the extracardiac catheter 120 is configured to be disposed in the esophagus of a patient when the system/device is in use.

The pulse generator 150 is configured to generate ablative pulse/energy, or referred to as electroporation pulse/energy, to be delivered to electrodes of the catheter pair 105. The electroporation pulse is typically high voltage and short pulse. The controller 140 is configured to control functional aspects of the electroporation ablation system/device 100. In embodiments, the electroporation controller 140 is configured to control the pulse generator 150 on the generation and delivery of ablative energy to electrodes of the intracardiac catheter 110 and the extracardiac catheter 120. In one embodiment, the intracardiac catheter 110 and the extracardiac catheter 120 each has one or more electrodes. In one case, each of the one or more electrodes of the intracardiac catheter 110 and the extracardiac catheter 120 is individually addressable. In such case, the controller 140 may control the ablative energy delivery to each electrode.

In some cases, the electroporation controller 140 receives sensor data collected by sensor(s) of catheter(s) and changes the ablative energy in response to the sensor data. In some cases, the electroporation controller 140 is configured to model the electric fields that can be generated by the catheter pair 105, which often includes consideration of the physical characteristics of the electroporation catheter pair 105 including the electrodes and spatial relationships of the electrodes on the electroporation catheter pair 105. In embodiments, the electroporation controller 140 is configured to control the ablative pulse to generate electric field among electrodes with field strength of no greater than 1500 volts per centimeter.

In embodiments, the electroporation catheter pair 105 allows electrical field to penetrate deeper into the ablation target wall (near-field bipolar) while avoiding skeletal muscle activation that is associated with unipolar (ablation catheter tip to skin electrode). In some cases, the intracardiac catheter 110 includes a first surface area and the extracardiac catheter 120 includes a second surface area, where the second surface area is different from the first surface area. In some cases, the second surface area is greater than the first surface area. As used herein, a surface area of a catheter refers to a sum of outer surface areas of electrodes of the catheter, where outer surfaces refer to surfaces of electrodes that will contact body mass and/or body fluid when in use. In some cases, the second surface area is greater than the first surface area by at least 10% of the first surface area. In some cases, the second surface area is greater than the first surface area by at least 20% of the first surface area. In some cases, the second surface area is greater than the first surface area by at least 30% of the first surface area. In some cases, the second surface area is greater than the first surface area by at least 50% of the first surface area. In embodiments, the extracardiac catheter 120 includes a temperature sensor. The temperature sensor is configured to detect a temperature in an extracardiac chamber when in use. In some cases, the temperature sensor is configured to detect a temperature in the esophagus when the electroporation ablation system/device 100 is in operation. In some cases, the controller is configured to reduce the ablative energy generated by the pulse generator 150 when the detected temperature is greater than a predetermined threshold.

In embodiments, the electroporation controller 140 includes one or more controllers, microprocessors, and/or computers that execute code out of memory 160, for example, non-transitory machine readable medium, to control and/or perform the functional aspects of the electroporation ablation system/device 100. In embodiments, the memory 160 can be part of the one or more controllers, microprocessors, and/or computers, and/or part of memory capacity accessible through a network, such as the world wide web. In embodiments, the memory 160 comprises a data repository 165, which is configured to store ablation data (e.g., location, energy, etc.), sensed data, modelled electric field data, treatment plan data, and/or the like.

In embodiments, the introducer sheath 130 is operable to provide a delivery conduit through which the intracardiac catheter 110 can be deployed to specific target sites at an intracardiac chamber. In embodiments, the other systems 170 includes an electro-anatomical mapping (EAM) system. In some cases, the EAM system is operable to track the location of the various functional components of the electroporation ablation system/device 100, and to generate high-fidelity three-dimensional anatomical and electro-anatomical maps of the cardiac chambers of interest. In embodiments, the EAM system can be the RHYTHMIA™ HDx mapping system marketed by Boston Scientific Corporation. Also, in embodiments, the mapping and navigation controller of the EAM system includes one or more controllers, microprocessors, and/or computers that execute code out of memory to control and/or perform functional aspects of the EAM system.

The EAM system generates a localization field, via a field generator, to define a localization volume about the heart, and one or more location sensors or sensing elements on the tracked device(s), e.g., the electroporation catheter pair 105, generate an output that can be processed by a mapping and navigation controller to track the location of the sensor, and consequently, the corresponding device, within the localization volume. In one embodiment, the device tracking is accomplished using magnetic tracking techniques, whereby the field generator is a magnetic field generator that generates a magnetic field defining the localization volume, and the location sensors on the tracked devices are magnetic field sensors.

In some embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller to track the location of the various location sensing electrodes within the localization volume.

In embodiments, the EAM system is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system utilizes the location information for the various tracked devices, along with cardiac electrical activity acquired by, for example, the electroporation catheter pair 105 or another catheter or probe equipped with sensing electrodes, to generate, and display via a display, detailed three-dimensional geometric anatomical maps or representations of the cardiac chambers as well as electro-anatomical maps in which cardiac electrical activity of interest is superimposed on the geometric anatomical maps. Furthermore, the EAM system can generate a graphical representation of the various tracked devices within the geometric anatomical map and/or the electro-anatomical map.

Embodiments of the present disclosure allows the electroporation ablation system/device 100 to be used for focal ablations and/or circumference ablations. The close proximity of the extracardiac catheter 120 can facilitate ablation with less or minimum muscle activation. In some cases, integrated with the EAM system, the system/device 100 allows graphical representations of the electric fields that can be produced by the electroporation catheter pair 105 to be visualized on an anatomical map of the patient and, in some embodiments, on an electro-anatomical map of the patient's heart.

According to embodiments, various components (e.g., the controller 140) of the electroporation ablation system 100 may be implemented on one or more computing devices. A computing device may include any type of computing device suitable for implementing embodiments of the disclosure. Examples of computing devices include specialized computing devices or general-purpose computing devices such “workstations,” “servers,” “laptops,” “desktops,” “tablet computers,” “hand-held devices,” “general-purpose graphics processing units (GPGPUs),” and the like, all of which are contemplated within the scope of FIG. 1 with reference to various components of the system 100.

In some embodiments, a computing device includes a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more busses (such as, for example, an address bus, data bus, or combination thereof). Similarly, in some embodiments, the computing device may include a number of processors, a number of memory components, a number of I/O ports, a number of I/O components, and/or a number of power supplies. Additionally, any number of these components, or combinations thereof, may be distributed and/or duplicated across a number of computing devices.

In some embodiments, the memory 160 includes computer-readable media in the form of volatile and/or nonvolatile memory, transitory and/or non-transitory storage media and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In some embodiments, the memory 160 stores computer-executable instructions for causing a processor (e.g., the controller 140) to implement aspects of embodiments of system components discussed herein and/or to perform aspects of embodiments of methods and procedures discussed herein.

Computer-executable instructions may include, for example, computer code, machine-useable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

The data repository 165 may be implemented using any one of the configurations described below. A data repository may include random access memories, flat files, XML files, and/or one or more database management systems (DBMS) executing on one or more database servers or a data center. A database management system may be a relational (RDBMS), hierarchical (HDBMS), multidimensional (MDBMS), object oriented (ODBMS or OODBMS) or object relational (ORDBMS) database management system, and the like. The data repository may be, for example, a single relational database. In some cases, the data repository may include a plurality of databases that can exchange and aggregate data by data integration process or software application. In an exemplary embodiment, at least part of the data repository 165 may be hosted in a cloud data center. In some cases, a data repository may be hosted on a single computer, a server, a storage device, a cloud server, or the like. In some other cases, a data repository may be hosted on a series of networked computers, servers, or devices. In some cases, a data repository may be hosted on tiers of data storage devices including local, regional, and central.

Various components of the system/device 100 can communicate via or be coupled to via a communication interface, for example, a wired or wireless interface. The communication interface includes, but not limited to, any wired or wireless short-range and long-range communication interfaces. The wired interface can use cables, umbilicals, and the like. The short-range communication interfaces may be, for example, local area network (LAN), interfaces conforming known communications standard, such as Bluetooth® standard, IEEE 802 standards (e.g., IEEE 802.11), a ZigBee® or similar specification, such as those based on the IEEE 802.15.4 standard, or other public or proprietary wireless protocol. The long-range communication interfaces may be, for example, wide area network (WAN), cellular network interfaces, satellite communication interfaces, etc. The communication interface may be either within a private computer network, such as intranet, or on a public computer network, such as the internet.

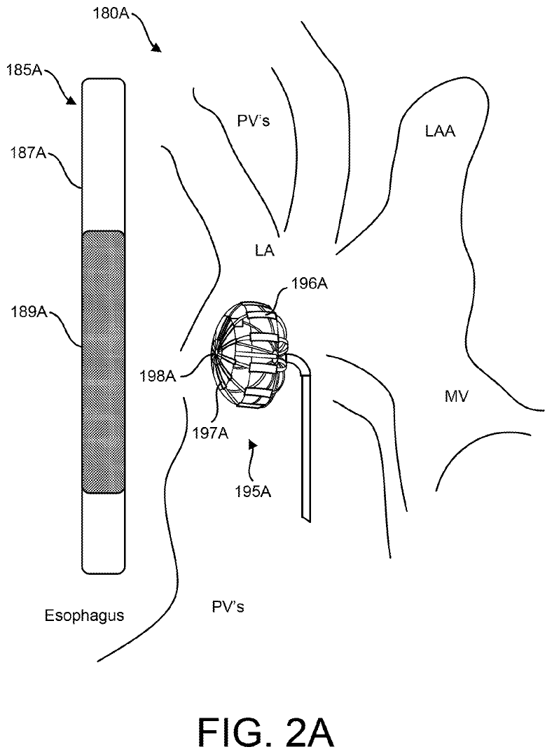

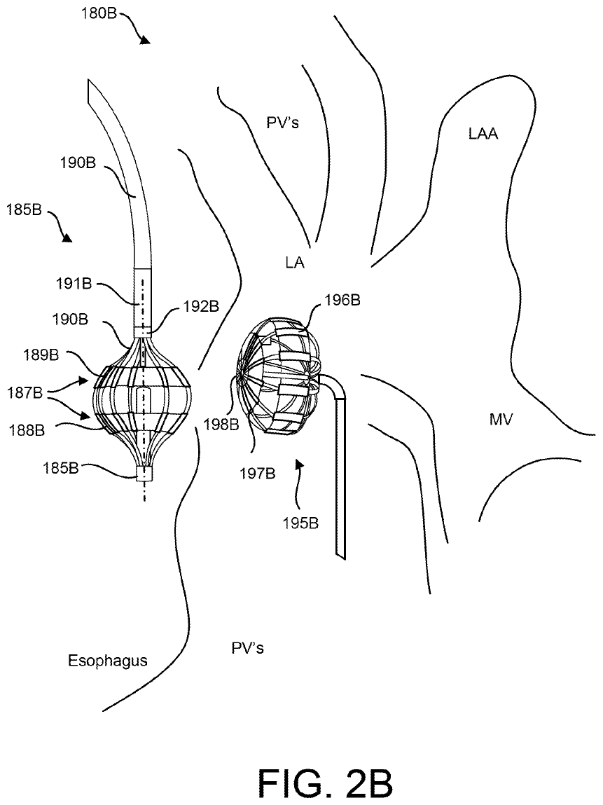

FIG. 2A depicts an illustrative view of an example of an electroporation ablation device 180A in operation, in accordance with embodiments of the subject matter of the disclosure. The electroporation ablation device 180A includes a first catheter 185A disposed in the esophagus of a patient and a second catheter 195A disposed in the intracardiac chamber of the patient. The first catheter 185A and/or the second catheter 195A may use any one of the configuration of electroporation catheters described herein. In one embodiment, the first catheter 185A is deflectable, as is generally known in the art. In the example illustrated, the first catheter 185A includes a catheter shaft 187A and an electrode 189A disposed on the catheter shaft 187A. The catheter shaft 187A can be constructed in any manner, whether now known or later developed, suitable for flexible catheters.

In embodiments, the electrode 189A can take on any form suitable for insertion into the esophagus. For example, in embodiments, the electrode 189A may be formed from a conductive coil similar to known construction techniques for shocking coils on intracardiac defibrillator leads. In other embodiments, the electrode 189A may be formed on a flexible circuit structure attached to or disposed on the catheter shaft 187A. Regardless of the construction technique used, the electrode 189A of the first catheter 185A is configured to provide a return path for the ablative energy.

In the example illustrated, the second catheter 195A includes a plurality of electrodes. In the example illustrated, the one or more electrodes of the second catheter 195A comprise a plurality of proximal electrodes 196A and a plurality of distal electrodes 197A, where the plurality of distal electrodes 197A are disposed closer to a distal end 198A of the second catheter 195A than the plurality of proximal electrodes 196A. In some cases, the electrodes of the first catheter 185A and/or the second catheter 195A are used to create monophasic or multiphasic pulses.

FIG. 2B depicts another illustrative view of another example of an electroporation ablation device 180B in operation, in accordance with embodiments of the subject matter of the disclosure. In this examples, the electroporation ablation device 180B includes a first catheter 185B disposed in the esophagus of a patient and a second catheter 195B disposed in the intracardiac chamber of the patient. The first catheter 185B and/or the second catheter 195B may use any one of the configuration of electroporation catheters described herein. In one embodiment, the first catheter 185B is deflectable. In the example illustrated, the first catheter 185B includes a plurality of electrodes 187B disposed on a plurality splines 190B. In one embodiment, the electrodes 187B includes a plurality of distal electrodes 188B and a plurality of proximate electrodes 189B, where the distal electrodes 188B are disposed closer to a distal end 186B of the first catheter 185B. In some cases, each of the electrodes 187B comprises a flex circuit. In some cases, the electrodes 187B can use conductive materials, such as, for example, a metal, a metal composite, carbon nanotubes composite, multilayer graphene, or the like.

In embodiments, the first catheter 185B, more specifically, the shaft 191B has a longitudinal axis 192B. As illustrated, the electrodes 187B of the first catheter 185B are disposed on the plurality of splines 190B. Also as illustrated, when the electroporation ablation device 180B is in operation, the plurality of splines 190B are configured to be expanded outward from the longitudinal axis 192B. In one embodiment, the first catheter 185B includes an inflatable balloon (not shown).

In embodiments, the electrodes 187B of the first catheter 185B are configured to provide a return path for the ablative energy. In the example illustrated, the second catheter 195B includes a plurality of electrodes. In the example illustrated, the one or more electrodes of the second catheter 195B comprise a plurality of proximal electrodes 196B and a plurality of distal electrodes 197B, and wherein the plurality of distal electrodes 197B are disposed closer to a distal end 198B of the second catheter 195B than the plurality of proximal electrodes 196B.

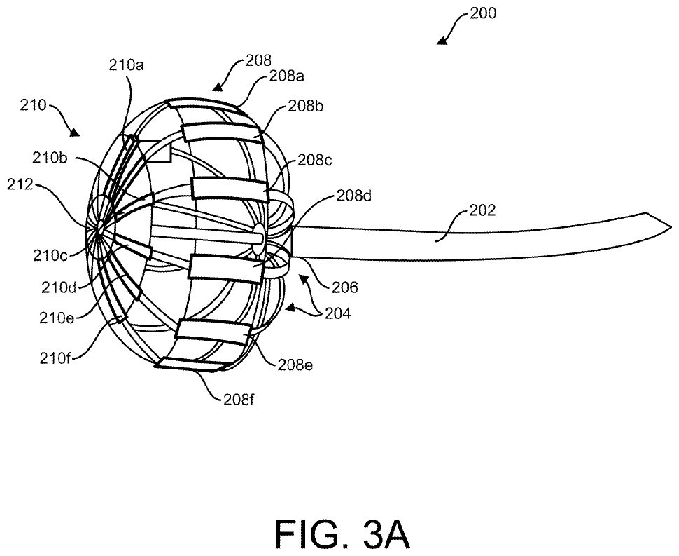

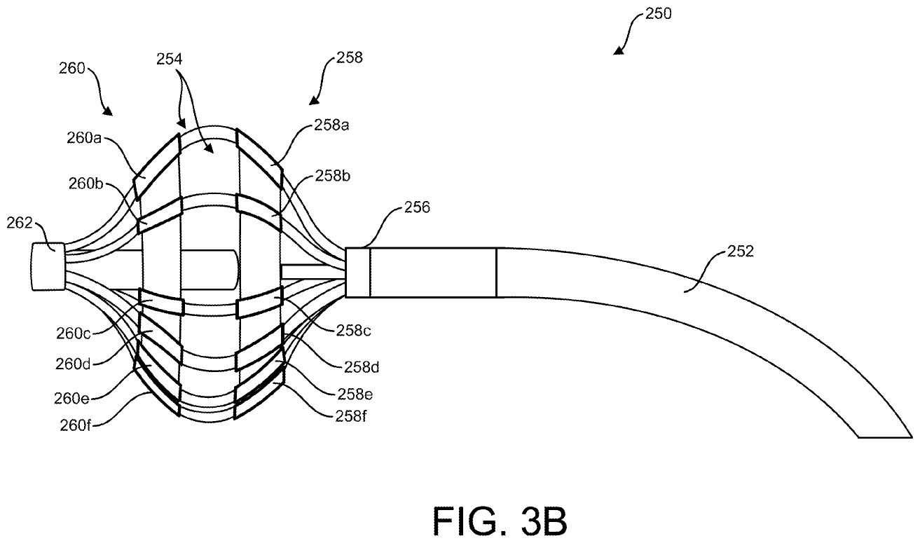

FIGS. 3A and 3B are diagrams illustrating example embodiments of catheters 200 and 250 that can be used for electroporation, including ablation by irreversible electroporation, in accordance with embodiments of the subject matter of the disclosure. The catheters 200 and 250 include electrodes, as described below, that are spaced apart from one another and configured to conduct electricity. Catheter characteristics are used to model electric fields that can be produced by the catheter. In embodiments, the characteristics used to model the electric fields can include: the type of catheter, such as a basket catheter that has a constant profile after being opened and a spline catheter that has a variable profile, which can be opened and closed by degree; the form factor of the catheter, such as a balloon catheter, a basket catheter, and a spline catheter; the number of electrodes; the inter-electrode spacing on the catheter; the spatial relationships and orientation of the electrodes, especially in relation to other electrodes on the same catheter; the type of material that the electrodes are made of; and the shape of the electrodes. In embodiments, the type of catheter and/or the form factor of the catheter includes catheters, such as linear ablation catheters and focal ablation catheters. Where, the type of catheter and/or the form factor of the catheter is not limited to those mentioned herein.

FIG. 3A is a diagram illustrating the catheter 200, in accordance with embodiments of the subject matter of the disclosure. The catheter 200 includes a catheter shaft 202 and a catheter basket 204 connected to the catheter shaft 202 at the distal end 206 of the catheter shaft 202. The catheter basket 204 includes a first group of electrodes 208 disposed at the circumference of the catheter basket 204 and a second group of electrodes 210 disposed adjacent the distal end 212 of the catheter basket 204. Each of the electrodes in the first group of electrodes 208 and each of the electrodes in the second group of electrodes 210 is configured to conduct electricity and to be operably connected to a controller (e.g., the controller 140 in FIG. 1) and an ablative energy generator (e.g., the pulse generator 150 of FIG. 1). In embodiments, one or more of the electrodes in the first group of electrodes 208 and the second group of electrodes 210 includes metal.

Electrodes in the first group of electrodes 208 are spaced apart from electrodes in the second group of electrodes 210. The first group of electrodes 208 includes electrodes 208a-208f and the second group of electrodes 210 includes electrodes 210a-210f. Also, electrodes in the first group of electrodes 208, such as electrodes 208a-208f, are spaced apart from one another and electrodes in the second of electrodes 210, such as electrodes 210a-210f, are spaced apart from one another.

The spatial relationships and orientation of the electrodes in the first group of electrodes 208 and the spatial relationships and orientation of the electrodes in the second group of electrodes 210 in relation to other electrodes on the same catheter 200 is known or can be determined. In embodiments, the spatial relationships and orientation of the electrodes in the first group of electrodes 208 and the spatial relationships and orientation of the electrodes in the second group of electrodes 210 in relation to other electrodes on the same catheter 200 is constant, once the catheter is deployed.

As to electric fields, in embodiments, each of the electrodes in the first group of electrodes 208 and each of the electrodes in the second group of electrodes 210 can be selected to be an anode or a cathode, such that electric fields can be set up between any two or more of the electrodes in the first and second groups of electrodes 208 and 210. Also, in embodiments, each of the electrodes in the first group of electrodes 208 and each of the electrodes in the second group of electrodes 210 can be selected to be a biphasic pole, such that the electrodes switch or take turns between being an anode and a cathode. Also, in embodiments, groups of the electrodes in the first group of electrodes 208 and groups of the electrodes in the second group of electrodes 210 can be selected to be an anode or a cathode or a biphasic pole, such that electric fields can be set up between any two or more groups of the electrodes in the first and second groups of electrodes 208 and 210.

In embodiments, electrodes in the first group of electrodes 208 and the second group of electrodes 210 can be selected to be biphasic pole electrodes, such that during a pulse train including a biphasic pulse train, the selected electrodes switch or take turns between being an anode and a cathode, and the electrodes are not relegated to monophasic delivery where one is always an anode and another is always a cathode. In some cases, the electrodes in the first and second group of electrodes 208 and 210 can form electric fields with electrode(s) of another catheter. In such cases, the electrodes in the first and second group of electrodes 208 and 210 can be anodes of the fields, or cathodes of the fields.

Further, as described herein, the electrodes are selected to be one of an anode and a cathode, however, it is to be understood without stating it that throughout this disclosure the electrodes can be selected to be biphasic poles, such that they switch or take turns between being anodes and cathodes. In some cases, one or more of the electrodes in the first group of electrodes 208 are selected to be cathodes and one or more of the electrodes in the second group of electrodes 210 are selected to be anodes. Also, in embodiments, one or more of the electrodes in the first group of electrodes 208 can be selected as a cathode and another one or more of the electrodes in the first group of electrodes 208 can be selected as an anode. In embodiments, one or more of the electrodes in the second group of electrodes 210 can be selected as a cathode and another one or more of the electrodes in the second group of electrodes 210 can be selected as an anode.

FIG. 3B is a diagram illustrating the catheter 250, in accordance with embodiments of the subject matter of the disclosure. The catheter 250 includes a catheter shaft 252 and catheter splines 254 connected to the catheter shaft 252 at the distal end 256 of the catheter shaft 252. The catheter splines 254 includes a first group of electrodes 258 disposed proximal the maximum circumference of the catheter splines 254 and a second group of electrodes 260 disposed distal the maximum circumference of the catheter splines 254. Each of the electrodes in the first group of electrodes 258 and each of the electrodes in the second group of electrodes 260 is configured to conduct electricity and to be operably connected to the electroporation console (not shown). In embodiments, one or more of the electrodes in the first group of electrodes 258 and the second group of electrodes 260 includes metal.

Electrodes in the first group of electrodes 258 are spaced apart from electrodes in the second group of electrodes 260. The first group of electrodes 258 includes electrodes 258a-258f and the second group of electrodes 260 includes electrodes 260a-260f. Also, electrodes in the first group of electrodes 258, such as electrodes 258a-258f, are spaced apart from one another and electrodes in the second of electrodes 260, such as electrodes 260a-260f, are spaced apart from one another.

The spatial relationships and orientation of the electrodes in the first group of electrodes 258 and the spatial relationships and orientation of the electrodes in the second group of electrodes 260 in relation to other electrodes on the same catheter 250 are known or can be determined. In embodiments, the spatial relationships and orientation of the electrodes in the first group of electrodes 258 and the spatial relationships and orientation of the electrodes in the second group of electrodes 260 in relation to other electrodes on the same catheter 250 are variable, where the distal end 262 of the catheter 250 can be extended and retracted which changes the spatial relationships and orientation of the electrodes 258 and 260. In some embodiments, the spatial relationships and orientation of the electrodes in the first group of electrodes 258 and the spatial relationships and orientation of the electrodes in the second group of electrodes 260 on the same catheter 250 is constant, once the catheter 250 is deployed.

As to electric fields, in embodiments, each of the electrodes in the first group of electrodes 258 and each of the electrodes in the second group of electrodes 260 can be selected to be an anode or a cathode, such that electric fields can be set up between any two or more of the electrodes in the first and second groups of electrodes 258 and 260. Also, in embodiments, groups of the electrodes in the first group of electrodes 258 and groups of the electrodes in the second group of electrodes 260 can be selected to be an anode or a cathode, such that electric fields can be set up between any two or more groups of the electrodes in the first and second groups of electrodes 258 and 260. In some cases, the electrodes in the first and second group of electrodes 258 and 260 can form electric fields with electrode(s) of another catheter. In such cases, the electrodes in the first and second group of electrodes 258 and 260 can be anodes of the fields, or cathodes of the fields.

In some embodiments, one or more of the electrodes in the first group of electrodes 258 are selected to be cathodes and one or more of the electrodes in the second group of electrodes 260 are selected to be anodes. Also, in embodiments, one or more of the electrodes in the first group of electrodes 258 can be selected as a cathode and another one or more of the electrodes in the first group of electrodes 258 can be selected as an anode. In addition, in embodiments, one or more of the electrodes in the second group of electrodes 260 can be selected as a cathode and another one or more of the electrodes in the second group of electrodes 260 can be selected as an anode. Using the characteristics of the catheter 250 and the surrounding tissue, an electroporation controller (e.g., the controller 140 of FIG. 1) can determine models for the various electric fields that can be produced by the catheter 250.

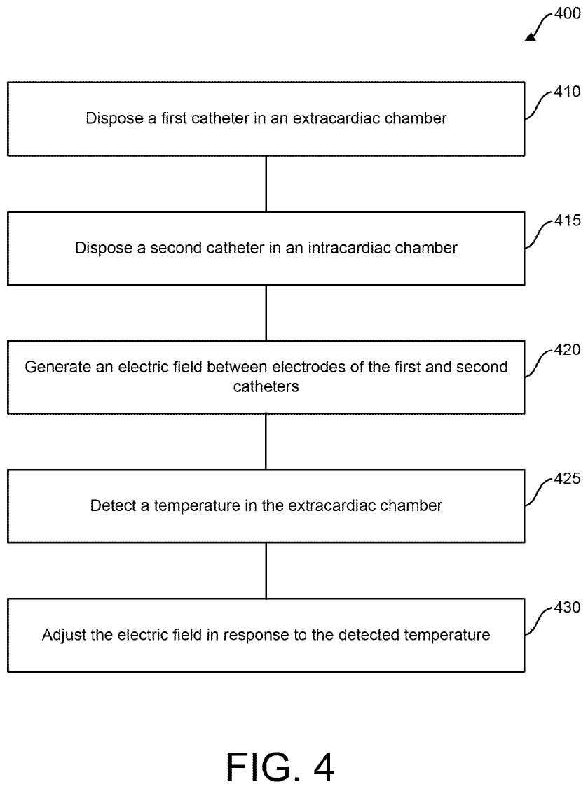

FIG. 4 is an example flow diagram depicting an illustrative method 400 of using an electroporation ablation device, in accordance with some embodiments of the present disclosure. Aspects of embodiments of the method 400 may be performed, for example, by an electroporation ablation system/device (e.g., the system/device 100 depicted in FIG. 1). One or more steps of method 400 are optional and/or can be modified by one or more steps of other embodiments described herein. Additionally, one or more steps of other embodiments described herein may be added to the method 400. First, the electroporation ablation system/device is configured to dispose a first catheter in an extracardiac chamber of a patient (410). In some cases, the first catheter is disposed in the esophagus of the patient. In embodiments, the first catheter is disposed anatomically approximate (e.g., a distance of less than 10 centimeters) to a target ablation location. The ablation system/device is also configured to dispose a second catheter of the electroporation ablation device in an intracardiac chamber of the patient (415). In embodiments, the second catheter is disposed approximate to the target ablation location. The first catheter and/or the second catheter may use any one of the configuration of electroporation catheters described herein.

In embodiments, the electroporation ablation system/device is configured to generate an electric field between electrodes of the first catheter and the second catheter (420). In one embodiment, the electric field has a field strength no higher than 1500 volts per centimeters. In embodiments, the electroporation ablation system/device is configured to detect a temperature in the extracardiac chamber (425). In some cases, the first catheter includes sensors such as, a temperature sensor, accelerometer, an impedance sensor, and/or the like. The electroporation ablation system/device is further configured to adjust the electric field (e.g., the electric field strength) in response to the detected temperature (430). In one embodiment, the electroporation ablation system/device is configured to reduce the electric field strength when the detected temperature greater than a predetermined threshold. In some cases, the first catheter is deflectable.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An electroporation ablation device, comprising:
a first catheter comprising one or more first electrodes and having a first surface area,
a second catheter comprising one or more second electrodes and having a second surface area,
wherein the first surface area is larger than the second surface area,
wherein when the electroporation ablation device is in operation for ablating a target tissue,
the first catheter is configured to be disposed in an esophagus and anatomically proximate to the target tissue,
the second catheter is configured to be disposed at an intracardiac location proximate to the target tissue, and
the electroporation ablation device is configured to generate an electric field between the one or more first electrodes and the one or more second electrodes with electric field strength sufficient to ablate the target tissue via irreversible electroporation.

2. The electroporation ablation device of claim 1, wherein the first catheter comprises a temperature sensor.

3. The electroporation ablation device of claim 2, wherein the temperature sensor is configured to detect a temperature in the esophagus when the electroporation ablation device is in operation.

4. The electroporation ablation device of claim 3, wherein the ablative energy is reduced when the detected temperature is greater than a predetermined threshold.

5. The electroporation ablation device of claim 1, wherein the first catheter is deflectable.

6. The electroporation ablation device of claim 1, wherein the one or more first electrodes are configured to provide a return path for the ablative energy.

7. The electroporation ablation device of claim 1, wherein the one or more second electrodes comprise a plurality of distal electrodes and a plurality of proximal electrodes, and wherein the plurality of distal electrodes are disposed closer to a distal end of the second catheter than the plurality of proximal electrodes.

8. The electroporation ablation device of claim 1, wherein the first surface area is larger than the second surface area by 10% of the second surface area.

9. The electroporation ablation device of claim 1, wherein the first catheter comprises an inflatable balloon.

10. The electroporation ablation device of claim 1, wherein the ablative energy is less than 1500 volts per centimeter.

11. A method using an electroporation ablation device, the method comprising:
disposing a first catheter of the electroporation ablation device anatomically approximate to a target ablation location in an extracardiac chamber, the first catheter comprising one or more first electrodes;
disposing a second catheter of the electroporation ablation device approximate to the target ablation location in an intracardiac chamber, the second catheter comprising one or more second electrodes; and
generating an electric field between the one or more first electrodes and the one or more second electrodes with electric field strength sufficient to ablate the target tissue via irreversible electroporation.

12. The method of claim 11, wherein the first catheter comprises a temperature sensor.

13. The method of claim 11, wherein the first catheter is disposed in the esophagus.

14. The method of claim 13, wherein the electric field strength is reduced when the detected temperature is greater than a predetermined threshold.

15. The method of claim 11, wherein the first catheter is deflectable.

16. An electroporation ablation system, comprising:
an electroporation ablation device comprising:
a first catheter comprising one or more first electrodes and having a first surface area, and
a second catheter comprising one or more second electrodes and having a second surface area,
a pulse generator configured to generate and deliver ablative energy to the electroporation ablation device, and
a controller coupled to the pulse generator and the electroporation ablation device,
wherein the first surface area is larger than the second surface area,
wherein when the electroporation ablation device is in operation for ablating a target tissue,
the first catheter is configured to be disposed in an esophagus and anatomically proximate to the target tissue, and
the second catheter is configured to be disposed at an intracardiac location proximate to the target tissue.

17. The electroporation ablation system of claim 16, wherein the electroporation ablation device is configured to generate an electric field between the one or more first electrodes and the one or more second electrodes with electric field strength sufficient to ablate the target tissue via irreversible electroporation.

18. The electroporation ablation system of claim 16, wherein the first catheter is deflectable.

19. The electroporation ablation system of claim 16, wherein the first surface area is larger than the second surface area by 10% of the second surface area.

20. The electroporation ablation system of claim 16, wherein the one or more first electrodes are configured to provide a return path for the ablative energy.
